(19) United States
(12) Patent Application Publication
(10) Pub. No.: US 2010/0112699 A1
(43) Pub. Date: May 6, 2010

(54) POROUS COMPOSITE BIOMATERIALS AND PRODUCTION METHOD OF THE SAME

(76) Inventors: Yu-Der LEE, Hsin Chu City (TW); Kuo-Yung Chang, Hsin Chu City (TW); Li-Wei Cheng, Hsin Chu City (TW)

Correspondence Address: Muncy, Geissler, Olds & Lowe, PLLC, P.O. BOX 1364, FAIRFAX, VA, 22038-1364, US

(21) Appl. No.: 12/344,931

(22) Filed: Dec. 29, 2008

(30) Foreign Application Priority Data
Oct. 30, 2008 (TW) 097141886

(51) Int. Cl. C12N 5/06; C07K 14/00; C08L 89/00

(52) U.S. Cl. 435/395; 530/395; 525/54.1

(57) ABSTRACT

The invention discloses a porous composite biomaterial comprising of poly(γ-glutamic acid)-g-chondroitin sulfate (γ-PGA-g-CS) copolymer and poly(ε-caprolactone). The composite biomaterial provides a three-dimensional microenvironment for using as a scaffold for tissue engineering and for supporting the attachment and proliferation of cells. The invention also discloses a method of producing a porous composite biomaterial.

### FIELD OF THE INVENTION

The present invention is generally related to the field of a porous composite biomaterial, more particularly, to a porous composite biomaterial of scaffold for tissue engineering.

### DESCRIPTION OF THE PRIOR ART

Though the medical science has progressed over the years, issues regarding tissue lesion or disability are mainly still managed by conventional surgical operations of organ transplants. Because of the problems of the source shortage and the immunological rejection, the development of tissue engineering is widely carried out attempting to solve the problems by culturing the human organs and tissues artificially for patients all over the world. The basic concept of tissue engineering is to develop the substitutes of the wounded tissues based on biological and engineering technologies and maintain the normal operations of one human body by acquiring a few cells from the human body, implanting the cells into the plane or the steric materials to culture more cells in vitro, and implanting the cultured cells into the human body to make up or replace the wounded tissues of the human body. Because the cells are sourced from the same human body, the immune responses may be avoided. The base material of the cell culture scaffold is important relating to the cell culture processes. The materials of the scaffold should not only provide the spaces for cells to attach and to culture, but also leave the structural messages to the cells to proliferate and differentiate to the needed type of cells. Therefore, the materials for culturing the cells of tissues should generally fulfill the following requirements:
1. No cytotoxicity and compatible with cells: It allows the cells to attach on the material and to proliferate.
2. High porosity: Without enough space, the cells can proliferate only on the surface of the material but not deep inside the material. For culturing the tissues having three-dimensional structures, it is best to use the three-dimensional materials with interconnected porous structures to facilitate the conveyance of the nutrition and the wastes related to the cells.
3. Appropriate mechanical strength: The material should at least be able to afford the weight of the new generated cartilage tissues and have the properties of the polymers which is easy to be processed to fit the different requirements of the patients.
4. Biodegradability: The material provides a temporary scaffold for cells to proliferate, but they should be biodegradable and be replaced by the new generated cells and the extracelluar matrices (ECM) of such cells to form a whole new tissue.

The base materials of making the scaffolds generally comprise the natural materials and the artificial synthetic materials, and owning their advantages and disadvantages, respectively. While the natural materials which acquired from the nature sources are quite compatible with the cells, their applications are limited due to their low degrade rate and mechanical strength. In contrast, the artificial synthetic materials have relative good mechanical strength and controllable degrade rate, but they have relative bad cell compatibility. The United States Patent Application 2004/0166169 “Porous Extracellular Matrix Scaffold and Method” discloses a material used for porous scaffold and the relative method, but it did not mention the copolymer disclosed in the present invention. The copolymer will be described in detail in this specification.

The above-mentioned natural materials include collagen, alginate, poly(γ-glutamic acid) (γ-PGA), chitosan, polysaccharides, etc. Among the variety of natural copolymers, poly(γ-glutamic acid) is a totally biodegradable natural copolymer. Its molecular weight is in the range of about 100 kDA to 1000 kDA. It has good biocompatibility, biodegradability, water absorption ability, and water permeability, so that it is a good biomedical material. The poly(γ-glutamic acid) is formed from the polymerization of the glutamic acids. Because it is comprised of unitary amino acid unit, it has the properties of non-toxicity, biocompatibility, and biodegradability. Besides, when the hydrogen atom on the “α-COOH” position of the poly(γ-glutamic acid) is displaced, its water absorption ability is improved, and it is contributive to increase the hydrophilicity of the scaffold made of poly(γ-glutamic acid). Adding the water solube carbodiimides as cross-linking agents, poly(γ-glutamic acid) and fiber glue are utilized to synthesize a new biological gel which has low cytotoxicity for human body. Furthermore, the complex salt of poly(γ-glutamic acid) (with magnesium, calcium, barium, sodium, lithium, etc) is widely applied in the field of biomedical material, such as the materials of the surgical suture line, the materials of wound care dressings, the materials of wound healings, and the materials of hemostasis. Chondroitin sulfate (CS), one of the common mucopolysaccharides in human body, is one of the components of the extracellular matrix of cartilaginous tissue. It has been proved effective in proliferating the cartilaginous tissue and inhibiting the immune inflammation, and it make the cartilaginous tissue which is without vessels and nerves take the nutrition in and metabolize the waste. The chondroitin sulfate, with its low degree of crystallinity, enables it to sustain the compression force and help to against the repeated stretching motions. Such natural polymers have good biocompatibility with cartilaginous cells and greatly improve the biological properties of the scaffold made of such materials.

There are various artificial synthetic polymeric materials, and they can be roughly divided into two groups, the biodegradable group and the non-biodegradable group. In biomedical applications, the biodegradable group is more important. After implanting the polymers into the human body, the polymers can be decomposed into non-toxic small molecules by the microorganisms or the enzymes in the human body and be filtered by the kidney or be excreted by the process of metabolism. Thus, the patients can avoid suffering multiple surgical operations and therefore reduce the pain. These polymers mainly comprise carbon chains, further comprising different structures such as ester bond, ether bond, amino group, etc. There are plenty of biodegradable materials, but when applying to biomedical field, the chosen materials should be degraded within an acceptable time range and be mainly small molecules after degrading. Thus, the chosen materials can easily be absorbed or decomposed by creatures, and they can be excreted into the environment and be recycled in the environment to reduce the impacts of the environment. Polyester polymers play a major part in biodegradable polymeric materials, because their ester bonds can easily be broken by hydrolysis to form lactic acids that can be absorbed by a creature, thereby transforming the lactic acids into carbon dioxides and water molecules via metabolism inside the body of creature, and then excreted from the creature. Therefore, such polyester polymers are widely used in the field of biomedical materials. Poly(ε-caprolactone) (PCL) is a common aliphatic polyester which is formed from the ring opening polymerization of ε-caprolactones. Poly(ε-caprolactone) has properties such as good mechanical strength, biocompatibility, biodegradability, and permeability. It is widely used in the fields of biomedical engineering and been certified by Food and Drug Administration (FDA) that it can be applied to human bodies. So, poly(ε-caprolactone) has high potential to be the base material of issue engineering. However, poly(ε-caprolactone) has some disadvantages such as poor hydrophilicity, low cell adsorption ability, and slow degradation rate, thereby having negative effects on cell proliferation to be the base material of tissue culture. The academia and industries have paid efforts to develop the high potential base materials based on the poly(ε-caprolactone), trying to keep the advantages of poly(ε-caprolactone) and mend the foregoing disadvantages. The U.S. Pat. No. 5,932,539 “Biodegradable Polymer Matrix for Tissue Repair” disclosed a biodegradable polymer material for tissue repairing. It disclosed the use of chondroitin sulfate and several polyamino acids, but it did not mention the copolymers for improving the properties of poly(ε-caprolactone).

### SUMMARY OF THE INVENTION

The present invention discloses a porous composite biomaterial for tissue engineering and production method of the same.

In one aspect of the present invention, a new copolymer is synthesized by way of chemical bonding. The copolymer includes poly(γ-glutamic acid)-g-chondroitin sulfate (γ-PGA-g-CS) copolymer which comprising poly(γ-glutamic acid) and chondroitin sulfate. Mixing the salt particle type poly(γ-glutamic acid)-g-chondroitin sulfate copolymer with poly(ε-caprolactone) to make poly(γ-PGA)-g-CS/PCL composite biomaterials, a material of scaffold with similar properties of the extracelluar matrices is obtained. The scaffold made of poly(γ-PGA)-g-CS/PCL composite biomaterials have good hydrophilicity, cell adsorption ability, and degradability. So, it should be a better scaffold material for tissue culture than only poly(ε-caprolactone).

The poly(γ-glutamic acid)-g-chondroitin sulfate copolymer according to the present invention includes segments of poly(γ-glutamic acid) and chondroitin sulfate, and it is produced by the cross-linking reaction between the mixed poly(γ-glutamic acid) and chondroitin sulfate with the existence of the cross-linking agents and organic solvents.

The method of producing the porous composite biomaterial according to the present invention comprises mixing and dissolving the poly(γ-glutamic acid)-g-chondroitin sulfate copolymer and poly(ε-caprolactone) in solvents to get a solution, drying the solution and shaping, and getting the porous composite biomaterial. The weight percentage of the poly(γ-glutamic acid)-g-chondroitin sulfate copolymer in this biomaterial is in the range of about 1% to 70%, and the properties of the biomaterial changes with the different weight percentage of the poly(γ-glutamic acid)-g-chondroitin sulfate copolymer.

The porous composite biomaterial according to the present invention has improved hydrophilicity, cell adsorption ability, and degradability, which is better than only poly(ε-caprolactone) material.

### BRIEF DESCRIPTION OF THE DRAWINGS

FIG. 1 illustrates the diagram of the reaction mechanism of synthesizing the poly(γ-glutamic acid)-g-chondroitin sulfate copolymer of the preferred embodiment of the present invention.

FIG. 2 illustrates the ¹H-NMR diagram of the poly(γ-glutamic acid)-g-chondroitin sulfate copolymer of the preferred embodiment of the present invention.

FIG. 3 illustrates the corresponding structure diagram of FIG. 2.

FIG. 4 illustrates the ESCA diagram of the scaffold made of the porous composite biomaterial of the preferred embodiment of the present invention.

FIG. 5 illustrates the ESCA diagram of the scaffold made of the porous composite biomaterial of the preferred embodiment of the present invention.

FIG. 6 illustrates the ESCA diagram of the scaffold made of the porous composite biomaterial of the preferred embodiment of the present invention.

FIG. 7 illustrates the TEM diagram of the scaffold made of the porous composite biomaterial of the preferred embodiment of the present invention.

FIG. 8 illustrates the diagram of the weight loss of the various scaffolds via hydrolysis of the preferred embodiment of the present invention.

FIG. 9 illustrates the analysis diagram of the cytotoxicity of the various scaffolds of the preferred embodiment of the present invention.

### DESCRIPTION OF THE PREFERRED EMBODIMENT

The invention discloses a new poly(γ-glutamic acid)-g-chondroitin sulfate copolymer produced by chemical synthesis processes which graft the poly(γ-glutamic acid) onto the chondroitin sulfate to form a graft copolymer. The chondroitin sulfate, a natural polysaccharide polymer, is relatively abundant around the cartilage in the human body, and it plays the key role in induction of the chondrocytes (cartilage cells). And then, because poly(γ-glutamic acid) owns great hydrophilicity, the poly(γ-glutamic acid) is mixed with hydrophobic poly(ε-caprolactone) for the good of combining both their advantages in applying to the cartilage tissue culture in the field of tissue engineering. Combining the chondroitin sulfate, the major component of the extracellular matrix of cartilage tissue cells, into the composite biomaterial to form the three-dimensional porous scaffold, the scaffold is obtained and it provides a similar microenvironment to the original cell. Then, a faster cell proliferating rate and more extracellular matrices on the scaffold is obtained when culturing chondrocytes.

Followings are the exemplary preferred embodiments of the present invention:

Preparation of poly(γ-glutamic acid)-g-chondroitin sulfate copolymer

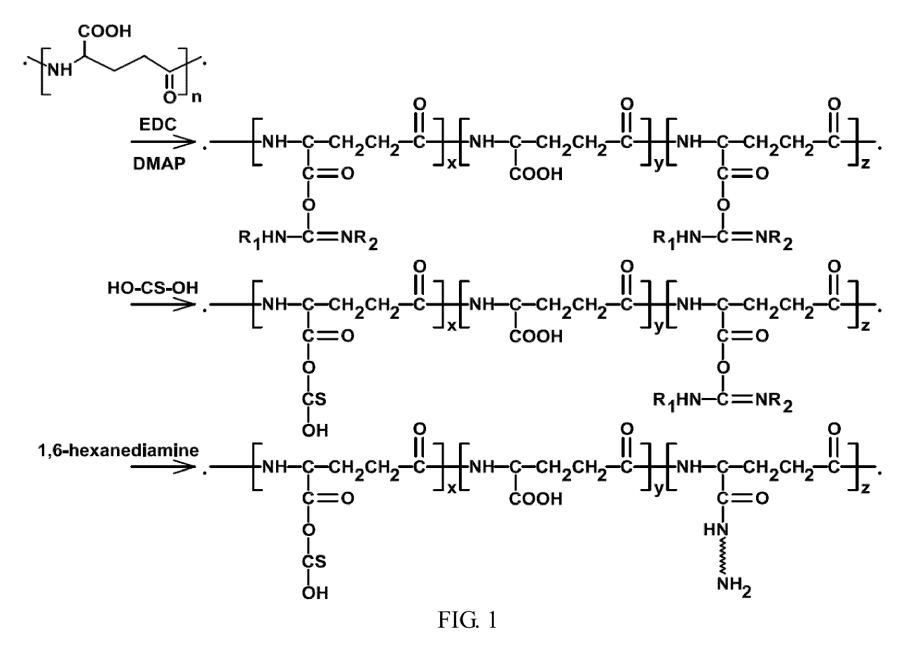

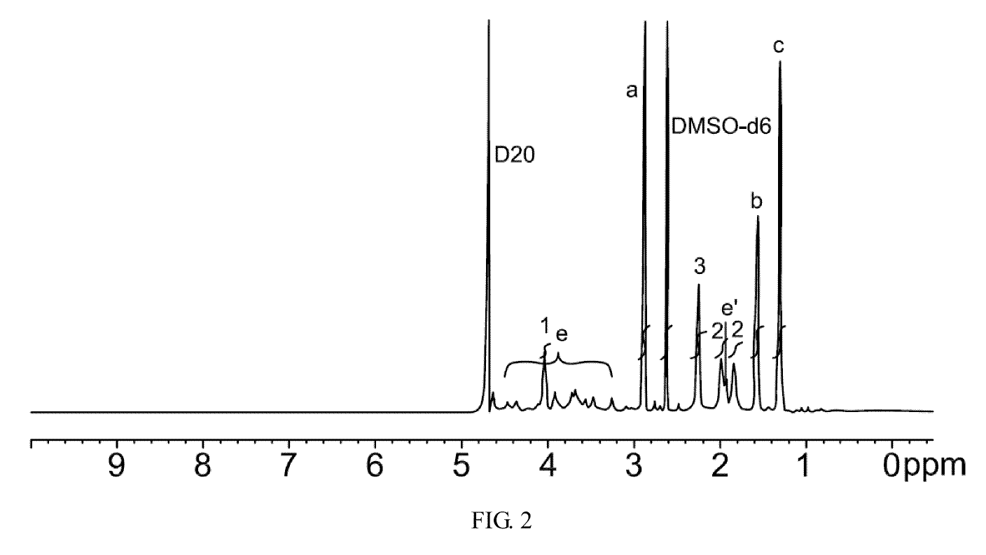

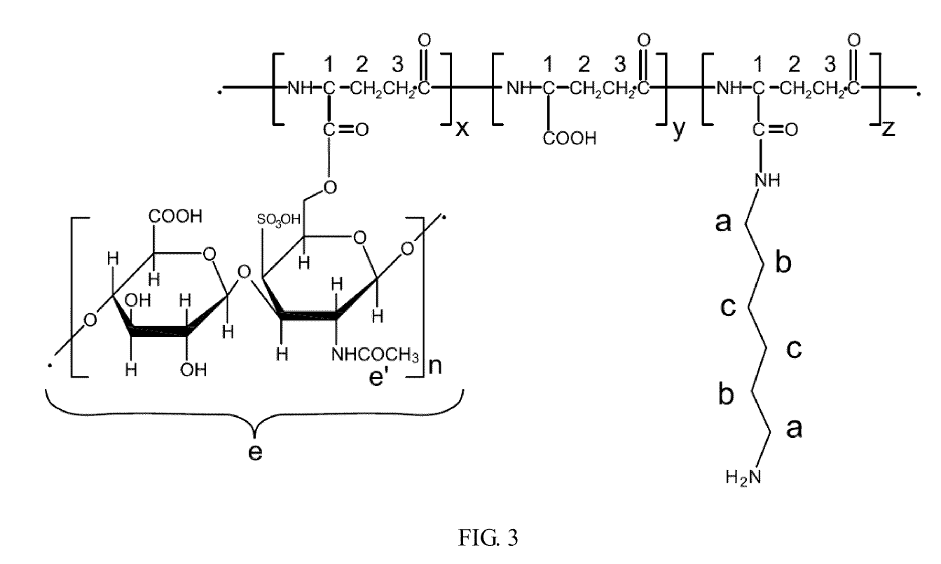

In this embodiment of the present invention, poly(γ-glutamic acid) (γ-PGA), 4-dimethylaminopyridine (DMAP) and 1-ethyl-3-(3-dimethylaminopropyl)carbodiimide (EDC) are put into dimethyl sulfoxide (DMSO) as a solution, and the solution is oscillated within the ultrasonic oscillator. Wherein the EDC can be replaced by N,N′-dicyclohexylcarbodiimide (DCC). On the other hand, adequate amount of chondroitin sulfates are dissolved in the water as another solution. Then the two solutions are mixed, and the range of the ratio of dimethyl sulfoxide solution to water solution is as 5:5 to 9:1. Wherein the molar ratio of poly(γ-glutamic acid), chondroitin sulfate, and 1-ethyl-3-(3-dimethylaminopropyl)carbodiimide is 1:0.5:1.5. The solution is put in the sample bottle and stirred periodically, wherein the range of the period is between 1 to 48 hours. After the solution is added into excess of acetone with instillation, the product of precipitation is extracted by aspirator filtration. The precipitation is dissolved into phosphate buffer solution (PBS), wherein the molecular weight cut-off is in the range of 10,000 to 100,000. It is dialyzed in the deionized water for two days, and the water is changed every twelve hours. Then the product is obtained after dialysis. The following steps include putting the product in the centrifuge bottle and removing the liquid by lyophilization method, then a drying sequence is performed to dry the dry product. Then, the surface modification is performed on the product, wherein the surface modification comprises poly(γ-glutamic acid) and chondroitin sulfate with 1,6-hexanediamine. First, overdose of 1,6-hexanediamine is added into the solution to react under room temperature around 1 to 48 hours. The solution after reacting is put into the dialyzing membrane, wherein the molecular weight cut-off is 3,500 to 100,000. The solution is dialyzed in the deionized water for two days, and the water is changed every 12 hours. Then the product is obtained after dialysis. The reaction mechanism of the above-mentioned graft copolymerization is shown in FIG. 1. And then the nuclear magnetic resonance (NMR) is used to verify its structure, as shown in FIG. 2. The NMR diagram shows that the chondroitin sulfate has been successfully grafted onto the poly(γ-glutamic acid) via its hydroxyl group and formed poly(γ-glutamic acid)-g-chondroitin sulfate copolymer. FIG. 3 illustrates the structure and labels corresponding to ¹H-NMR diagram in FIG. 2. Furthermore, regarding to the above-mentioned copolymer, the average molecular weight of chondroitin sulfate is in the range of about 2,000 to 50,000, and the average molecular weight of poly(γ-glutamic acid) is in the range about 2,000 to 500,000.

### Preparation of Porous Scaffold

Due to the good hydrophilicity of poly(γ-glutamic acid) and chondroitin sulfate, above material is mixed with hydrophobic poly(ε-caprolactone) and get the scaffold which provides a similar microenvironment to the original cell. Faster cell proliferating rates are achieved and more extracellular matrices are generated on the scaffold in the processes of chondrocyte culture. In this embodiment of the present invention, an appropriate amount of copolymer is taken and dissolved into cosolvents (the range of the ratio of water and dimethyl sulfoxide is 5:5 to 9:1). Stirring the solution with high speed, subsequently, the chloroform is added into the solution. The appropriate amount of poly(ε-caprolactone) is dissolved into the solution to prepare various polymer solution with weight percentage of about 15%. When homogeneously dissolved, the sieved salts is put with the diameter 100 to 450 μm into the solution rapidly and stir it thoroughly. After that, the solution is poured into a Teflon mold for shaping and drying, and it is put into cosolvent (the range of the ratio of water and dimethyl sulfoxide is 5:5 to 9:1) with stirring. The solvent is changed every 12 hours till all the salts are removed, following by freezing and drying it to get the material. Subsequently, the excess part of the material is cut and then the scaffold for tissue culture is obtained.

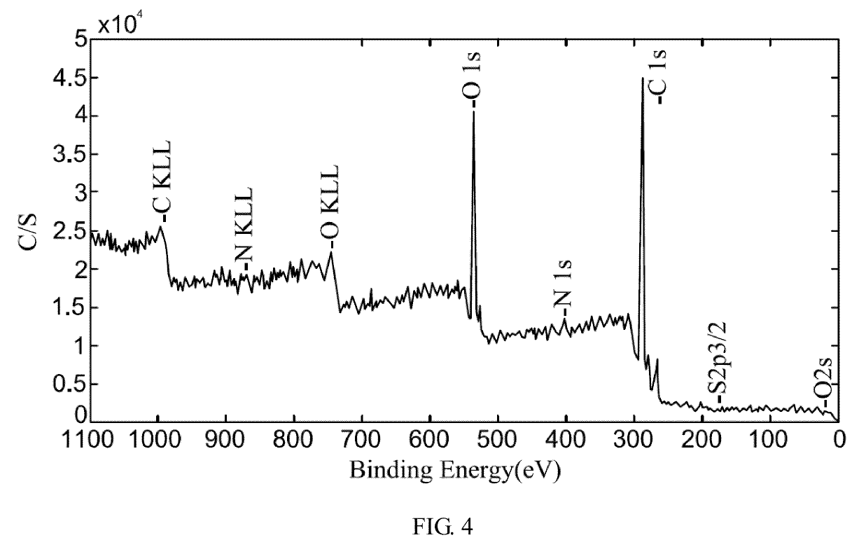

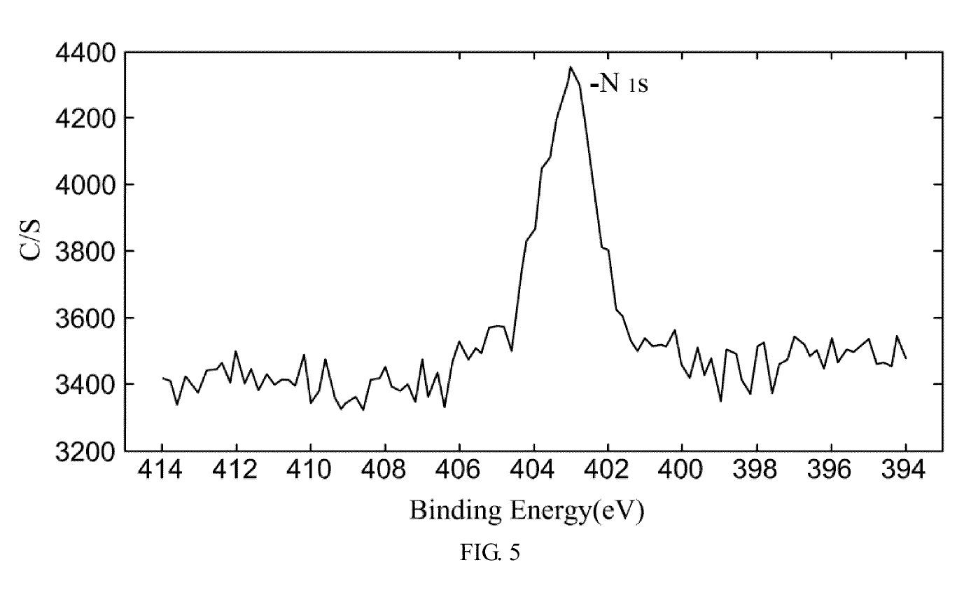

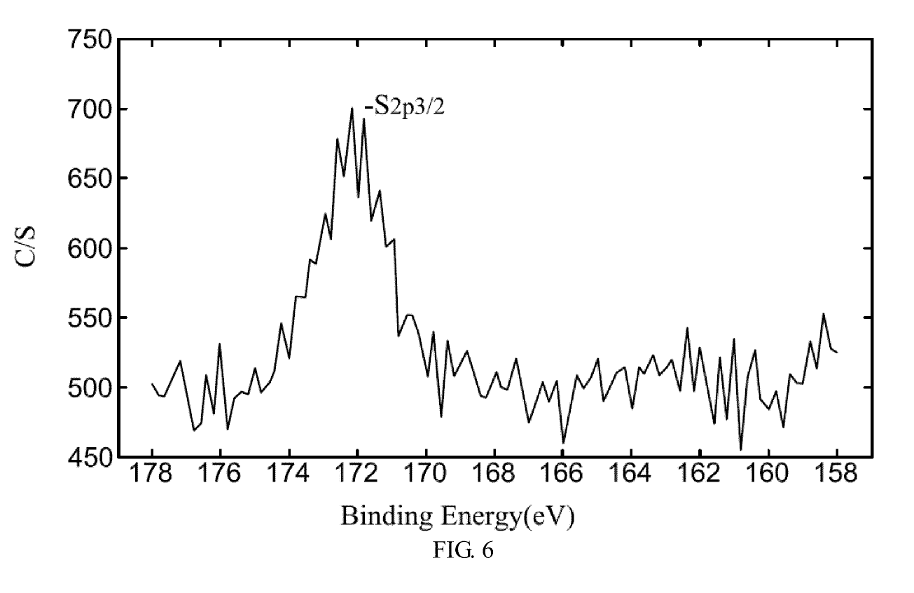

The electron spectroscopy for chemical analysis (ESCA) is introduced to measure and verify the structure of the above-mentioned porous scaffold. The ESCA emits X ray to excite and ionize the electrons from the inner-shell orbitals of the atoms of the surface of object to get photoelectrons. By determining the kinetic energies of these photoelectrons, the electron binding energies is obtained. The electron binding energies are dependent on the different properties of the atoms, making ESCA useful to identify the species and chemical states of the surface of object. ESCA is applied to verify the scaffold made of the porous materials of the present invention to check if there are actually the wanted copolymers inside the material. The result of ESCA verified the scaffold made of porous material contains poly(ε-caprolactone) (—C_{1S}, 283 eV; —O_{1S}, 531 eV; O KLL, 997 eV), poly(γ-glutamic acid) (—N_{1S}, 403 eV), and chondroitin sulfate (—S_{2P3,2}, 172.2 eV). The results are shown in FIG. 4, FIG. 5, and FIG. 6.

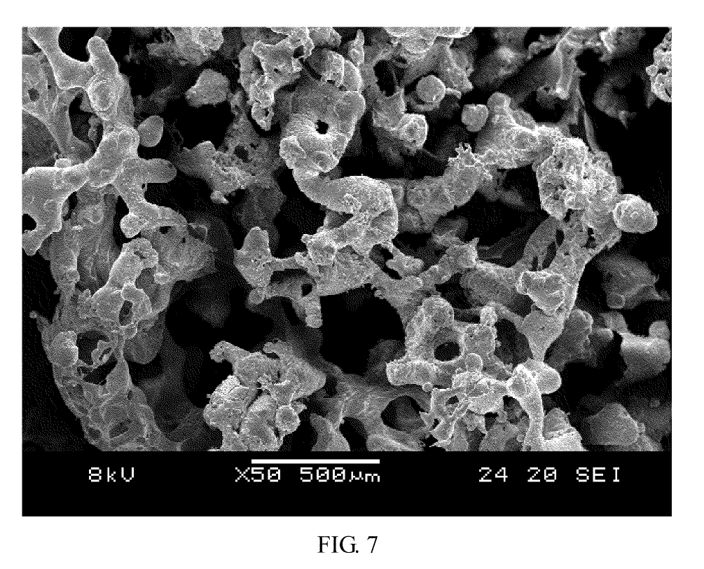

In addition, the field emission scanning electron microscope (FE-SEM) is utilized to observe the surface structure of the scaffold made of porous material, and the result is shown in FIG. 7.

### Analysis of the Mechanical Properties of the Scaffold

In another embodiment of the present invention, the scaffold originally immersed in the cell culture broth is taken out. Then the excess moisture on the scaffold is wiped off, and the scaffold is put on the center of the platform of compression mold. Then, with the compression rate of one millimeter per minute, the Universal Tensile Tester (Instron®) is used to commence the compression test. After inputting the required parameters, the compression modulus of the scaffold is gotten as shown in Table 1. From the experimental data, it is obvious that the compression strength of the scaffold decreases as the poly(γ-glutamic acid)-g-chondroitin sulfate copolymer content increases. This is due to the difference of molecular weights between the poly(γ-glutamic acid)-g-chondroitin sulfate copolymer and poly(ε-caprolactone) in the scaffold of the present invention. Therefore, the whole compression strength decreases as the poly(γ-glutamic acid)-g-chondroitin sulfate copolymer content in this scaffold increases.

TABLE 1

| | γ-PGA-g-CS | PCL content | Compression |
|---|---|---|---|
| Sample | content (%) | (%) | Strength (kPa) |
| PCL | — | 100 | 313.3 ± 58.6 |
| R10P90 | 10 | 90 | 93.3 ± 5.8 |
| R30P70 | 30 | 70 | 23.3 ± 15.3 |

Analysis of Degradability of the Scaffold made of Porous Materials

In this embodiment of the present invention, the degradability of the prepared scaffold made of porous material is analyzed via the percentage of weight change. At first, the dry weight W₀ of the scaffold is measured, and then the scaffold is put into a phosphate buffer solution at 37° C. for hydrolytic reaction and changing the buffer solution every three days. The scaffold is taken out of the solution periodically for weighting. Before weighting, the scaffold is put into the double distilled water with ultrasonic oscillation to remove the residue of salts from the buffer solution, and then the scaffold is dehydrated with the absolute alcohol and measure the weight at that time W₁. Then, the percentage of weight change is

$$\frac{( {W}_{0} - {W}_{1} )}{{W}_{0}} \times 100 \% .$$

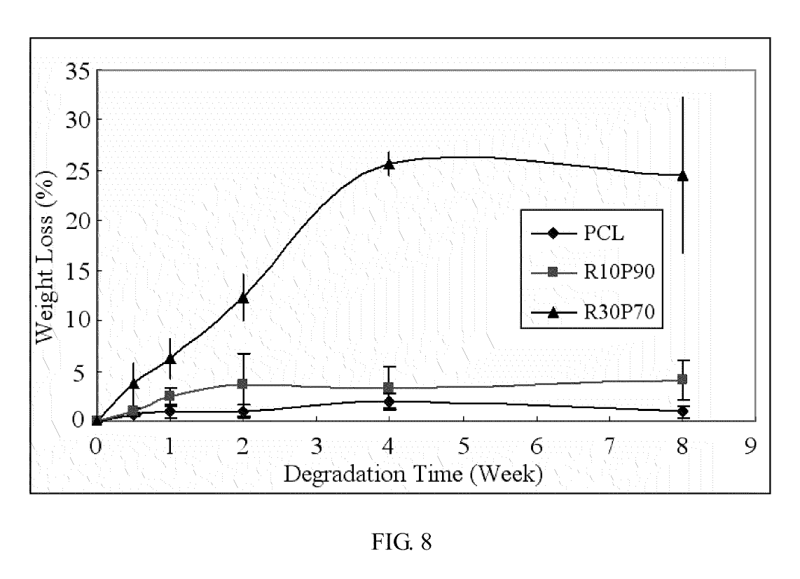

The result is shown in FIG. 8, wherein the poly(ε-caprolactone) content in the sample “PCL” is 100%, while the ratio of poly(γ-glutamic acid)-g-chondroitin sulfate copolymer and poly(ε-caprolactone) in “R10P90” is 10:90, and the ratio is 30:70 in “R30P70”. From the experimental result, it may be concluded that the degradability of scaffold increases as the poly(γ-glutamic acid)-g-chondroitin sulfate copolymer content increases and proved that the degradability of the scaffold may be improved by adding the poly(γ-glutamic acid)-g-chondroitin sulfate copolymer.

### The Water Absorption and the Porosity Test

In this embodiment of the present invention, the tests for water absorption and the porosity of the scaffold are applied. At first, the dry weight of the scaffold is measured as W₀, and then the scaffold is soaked in the double distilled water overnight. After that, the scaffold is taken out from the water, and the moisture on its surface is wiped off. Them the weight is measured as W₁. Then, the water absorption is

$$\frac{( {W}_{1} - {W}_{0} )}{{W}_{0}} \times 100 \% .$$

In the other hand, the measurement of porosity is based on Archimedes' Principle and use the pycnometer. At first, the pycnometer is made full of water, and then the weight of above pycnometer is measured as W₁. In the other hand, the weight of the scaffold is measured as W_{S}. Subsequently, the scaffold is put into the pycnometer, and the air is made out of the scaffold. After the pycnometer is refilled with water, the weight is measured as W₂. After that, the scaffold that is full of water is taken out of the pycnometer and the weight of the pycnometer at that time is measured as W₃. Then, the porosity (ε) is

$${V}_{P} = \frac{( {W}_{2} - {W}_{3} - {W}_{S} )}{{ρ}_{W}}$$

$${V}_{S} = \frac{( {W}_{1} - {W}_{2} + {W}_{S} )}{{ρ}_{W}}$$

$$ɛ = \frac{{V}_{P}}{( {V}_{P} + {V}_{S} )} = \frac{( {W}_{2} - {W}_{3} - {W}_{S} )}{( {W}_{1} - {W}_{3} )} ,$$

wherein V_{P} is the volume of the pore, V_{S} is the volume of scaffold, and ρ_{W} is the density of water. The result is shown as Table 2.

TABLE 2

| | γ-PGA-g-CS | PCL content | Water | |
|---|---|---|---|---|
| Sample | content (%) | (%) | absorption (%) | Porosity (%) |
| PCL | — | 100 | 352 ± 68 | 71 ± 3 |
| R10P90 | 10 | 90 | 662 ± 32 | 88 ± 3 |
| R30P70 | 30 | 70 | 740 ± 51 | 85 ± 4 |

The experimental result shows that the water absorption and the porosity of the scaffold both increase as the poly(γ-glutamic acid)-g-chondroitin sulfate copolymer content increases. This approves that adding poly(γ-glutamic acid)-g-chondroitin sulfate copolymer improves the water absorption and increase the porosity of the scaffold.

### Cytotoxicity Test

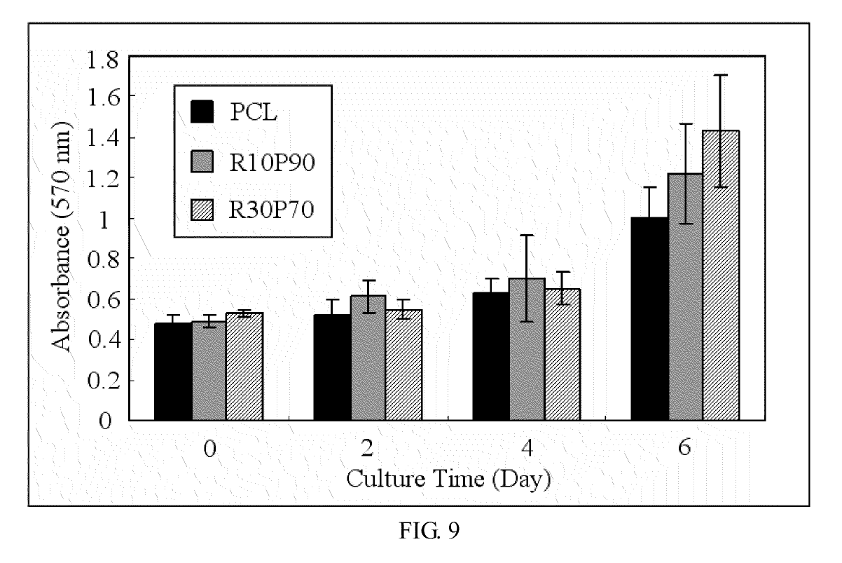

In this embodiment of the present invention, the cytotoxicity test is performed on the scaffold made of the porous composite biomaterials. First, the scaffold is soaked in 70% alcohol. Then the scaffold is put on the sterile bench and irradiated with ultraviolet ray for 22 hours. Subsequently, the scaffold is washed and soaked with phosphate buffer solution for three times under the radiation of ultraviolet ray and put on the ninety-six wells culture plate. After that, the 3T3 fibroblasts are inoculated onto the scaffold inside the ninety-six wells culture plate with the inoculum density of 2×10⁵ cells/scaffold and standed 3 hours for the attachment of the cells onto the scaffold. Then the scaffold is moved to a twelve wells culture plate, and adequate amount of cell culture brothes are added into the plate. Subsequently, the plate is put into the incubator for cell culture in 37° C., and the cell culture brothes are changed every two days. The cell culture brothes are removed at the initial day and the every second day. Then the scaffold is washed with phosphate buffer solution, and 40 μl/well dimethyl sulfoxide is added into it. When dissolving completely, 200 μl of the solution is taken into the ninety six wells culture plate, and its absorbance at the wavelength of 570 nm is recorded. The result is shown as FIG. 9 which verifies the scaffold made of the porous composite biomaterial of the present invention is non-cytotoxic.

### Analysis of Glycosaminoglycan (GAG) and Collagen

In this embodiment of the present invention, the absorbance properties of the complex for forming by pigments and glycosaminoglycan (GAG) is utilized to make quantitative analysis, wherein the glycosaminoglycan is a saccharide binding around the collagen. At first, the chondrocytes are decomposed in the scaffold by papain, and then they are dyed with 1,9-dimethylmethylene. The spectrophotometer is used then to determine the contents which are usually the indicator of the quality of the artificial cartilage. The applied method comprising hydrolyzing the collagen with the existence of strong acid in high temperature; releasing the hydroxyproline; dyeing with pigments; radiate with 550 nm ray; recording the absorbance and ploting the standard curve; and calculating the concentration of glycosaminoglycan and collagen. The result is shown as Table 3.

TABLE 3

| | Copolymer | PCL | GAG from the cells | | Collagen from the cells | |
|---|---|---|---|---|---|---|
| | content | content | μg/scaffold | | μg/scaffold | |
| sample | (%) | (%) | Two weeks | Four weeks | Two weeks | Four weeks |
| PCL | — | 100 | 3.75 ± 2.35 | 20.32 ± 5.58 | 3.95 ± 2.14 | 8.90 ± 1.14 |
| R10P90 | 10 | 90 | 12.61 ± 3.89 | 23.00 ± 3.38 | 5.44 ± 0.99 | 13.59 ± 6.57 |
| R30P70 | 30 | 70 | 28.21 ± 3.25 | 43.07 ± 3.09 | 10.21 ± 3.47 | 15.32 ± 4.74 |

The copolymer shown in Table 3 means the poly(γ-glutamic acid)-g-chondroitin sulfate copolymer of the present invention. The experimental result indicates that the glycosaminoglycan and the collagen in the scaffold increase as time goes by. Then, comparing with the samples of PCL, R10P90, and R30P70, it may be concluded that the more the poly(γ-glutamic acid)-g-chondroitin sulfate copolymer contained in the samples, the more the GAG and collagen secreted by cells. And this will make the cells more active outside the human bodies.

In conclusion, the scaffold, made of the porous composite biomaterials comprising poly(γ-glutamic acid)-g-chondroitin sulfate copolymer and poly(ε-caprolactone), owns greater hydrophilicity, cell adsorption ability, and degradability than the scaffold made of only poly(ε-caprolactone). And further, the cell culture of chondrocytes on the porous composite biomaterial also performs better than only poly(ε-caprolactone), and filling the requirements more for producing artificial cartilages in the field of biomaterial.

Embodiments of the invention are illustrated by way of example, and not by way of limitation. The scope of the present invention should be determined by the claims below. One skilled in the art may make further modifications and adaptations without departing from the basic scope of the present invention. Thus the modifications and adaptations are belonging to the spirit of the teaching here and should be included among the following claims.

What is claimed is:

1. A copolymer, comprising:
poly(γ-glutamic acid) (γ-PGA); and
chondroitin sulfate (CS);
wherein said copolymer is synthesized by cross-linking reaction via a cross-linking agent.

2. The copolymer of claim 1, wherein weight percentage of said poly(γ-glutamic acid) in said copolymer is in range of 1% to 50%, and weight percentage of said chondroitin sulfate in said copolymer is in range of 1% to 50%.

3. The copolymer of claim 1, wherein molar ratio of said poly(γ-glutamic acid) to said chondroitin sulfate is about 1:0.5.

4. The copolymer of claim 1, wherein said cross-linking agent includes 1-ethyl-3-(3-dimethylaminopropyl)carbodiimide (EDC) or N,N′-dicyclohexylcarbodiimide (DCC), and weight percentage of said cross-linking agent is in range of 1% to 200%.

5. The copolymer of claim 4, wherein molar ratio of said cross-linking agent to said poly(γ-glutamic acid) is about 1:1.5.

6. A porous composite biomaterial, comprising:
a copolymer; and
poly(ε-caprolactone) (PCL);
wherein weight percentage of said copolymer in said porous composite biomaterial is in range of 1% to 70%, and said copolymer is synthesized by cross-linking reaction between poly(γ-glutamic acid) and chondroitin sulfate via a cross-linking agent.

7. The porous composite biomaterial of claim 6, wherein weight percentage of said poly(γ-glutamic acid) in said copolymer is in range of 1% to 50%, and weight percentage of said chondroitin sulfate in said copolymer is in range of 1% to 50%.

8. The porous composite biomaterial of claim 6, wherein said cross-linking agent includes 1-ethyl-3-(3-dimethylaminopropyl)carbodiimide (EDC) or N,N′-dicyclohexylcarbodiimide (DCC), and weight percentage of said cross-linking agent is in range of 1% to 200%.

9. The porous composite biomaterial of claim 6, wherein said porous composite biomaterial may be utilized for scaffold of chondrocyte culture.

10. The porous composite biomaterial of claim 6, wherein hydrophilicity of said porous composite biomaterial increases as the content of said copolymer increases.

11. The porous composite biomaterial of claim 6, wherein adsorption ability of cells and tissues to said porous composite biomaterial increases as content of said copolymer increases.

12. The porous composite biomaterial of claim 6, wherein degradability of said porous composite biomaterial increases as content of said copolymer increases.

13. A method of producing porous composite biomaterial, which comprising:
cross-linking segments of poly(γ-glutamic acid) and chondroitin to synthesize a copolymer via a cross-linking agent;
forming a solution by dissolving and mixing said copolymer and poly(ε-caprolactone) in solvent; and
forming said porous composite biomaterial by drying and shaping said solution;
wherein weight percentage of said copolymer in said porous composite biomaterial is in range of 1% to 70%.

14. The method of producing porous composite biomaterial of claim 13, wherein weight percentage of said poly(γ-glutamic acid) in said copolymer is in range of 1% to 50%, and weight percentage of said chondroitin sulfate in said cpolymer is in range of 1% to 50%.

15. The method of producing porous composite biomaterial of claim 13, wherein said cross-linking agent includes 1-ethyl-3-(3-dimethylaminopropyl)carbodiimide (EDC) or N,N′-dicyclohexylcarbodiimide (DCC), and weight percentage of said cross-linking agent is in range of 1% to 200%.

16. The method of producing porous composite biomaterial of claim 13, further comprising producing scaffold for chondrocyte culture by said porous composite biomaterial.

17. The method of producing porous composite biomaterial of claim 13, wherein hydrophilicity of said porous composite biomaterial increases as the content of said copolymer increases.

18. The method of producing porous composite biomaterial of claim 13, wherein degradability of said porous composite biomaterial increases as content of said copolymer increases.

19. The method of producing porous composite biomaterial of claim 13, further comprising:
adding salts into said solution before drying and shaping said solution; and
removing said salts from said solution after drying and shaping said solution for forming three-dimensional porous structures of said porous composite biomaterial; wherein particle size of said salts is in range of 100 to 450 μm.

20. The method of producing porous composite biomaterial of claim 13, wherein said solvent includes water, dimethyl sulfoxide (DMSO), and chloroform.
